(19) United States
(12) Patent Application Publication
(10) Pub. No.: US 2026/0284383 A1
(43) Pub. Date: Sep. 24, 2026

(54) MULTI-CONTACT HELICAL WIRE STRUCTURE ELECTRODES AND CONNECTORS

(71) Applicant: NEURONOFF, INC., Cleveland, OH (US)

(72) Inventors: Stephan NIEUWOUDT, Cleveland, OH (US); Manfred Franke, Cleveland, OH (US)

(73) Assignee: NEURONOFF, INC., Cleveland, OH (US)

(21) Appl. No.: 19/456,700

(22) Filed: Jan. 22, 2026

Related U.S. Application Data
(63) Continuation-in-part of application No. 19/191,595, filed on Apr. 28, 2025.
(63) Continuation-in-part of application No. 18/835,430, filed on Aug. 2, 2024.
(63) Continuation-in-part of application No. 18/292,852, filed on Jan. 26, 2024.
Application No. 19/191,595 is a continuation-in-part of application No. 18/561,425, filed on Nov. 16, 2023.
Application No. 19/191,595 is a continuation-in-part of application No. 18/285,349, filed on Oct. 2, 2023, now Pat. No. 12,642,575.
Application No. 19/191,595 is a continuation-in-part of application No. 18/278,160, filed on Aug. 21, 2023.
(60) Provisional application No. 63/226,465, filed on Jul. 28, 2021.
(60) Provisional application No. 63/306,397, filed on Feb. 3, 2022.
(60) Provisional application No. 63/306,896, filed on Feb. 4, 2022.
(60) Provisional application No. 63/319,379, filed on Mar. 13, 2022.
(60) Provisional application No. 63/400,725, filed on Aug. 24, 2022.
(60) Provisional application No. 63/400,709, filed on Aug. 24, 2022.
(60) Provisional application No. 63/751,474, filed on Jan. 30, 2025.

(51) Int. Cl. A61N 1/05; A61N 1/372

(52) U.S. Cl. CPC A61N 1/0551; A61N 1/372

(57) ABSTRACT

A multi-contact wire structure electrode comprises a plurality of parallel strands of rope wire structures wound into a helical structure, two or more proximal stimulating electrodes formed by uncoated regions of the rope wire structures, and one or more distal connectors for electrical interfacing, wherein the distal connectors are configured for connection to an implantable pulse generator. A delivery system for inserting a partially helical wire structure electrode comprises an outer cannula configured to contain a partially helical wire structure electrode having a helical region and a non-helical region, and an inner sleeve positioned within the outer cannula configured to contain the non-helical region and extending to a transition region between the helical region and the non-helical region, wherein the inner sleeve mechanically stabilizes the non-helical region during insertion.

### CROSS-REFERENCE TO RELATED APPLICATIONS

This application is a utility patent application claiming priority to U.S. patent application Ser. No. 19/191,595, filed on Apr. 28, 2025, U.S. Provisional Patent Application Ser. No. 63/751,474, filed on Jan. 30, 2025, U.S. patent application Ser. Nos. 18/835,430 which was filed on Aug. 2, 2024, and 18/292,852 which was filed Jan. 26, 2024, all of which are hereby incorporated by reference in their entirety.

U.S. patent application Ser. No. 19/191,595 claims priority to U.S. patent application Ser. Nos. 18/561,425, filed Nov. 16, 2023, 18/285,349, filed Oct. 2, 2023, 18/278,160, filed Aug. 21, 2023, and is a U.S. National Stage of PCT/US2021/033007 filed on May 18, 2021, all of which are hereby incorporated by reference in their entirety.

U.S. patent application Ser. No. 18/835,430 claims priority to U.S. Provisional Patent Application Ser. No. 63/400,725 filed on Aug. 24, 2023 and is a U.S. National Stage of PCT/US23/012348 filed on Feb. 3, 2023, all of which are hereby incorporated by reference in their entirety.

U.S. patent application Ser. No. 18/292,852 is a U.S. National Stage of PCT/US2022/038719 which was filed Jul. 28, 2022, and claims priority to Provisional Patent Application Ser. No. 63/226,465, filed on Jul. 28, 2021, all of which are hereby incorporated by reference in their entirety.

PCT/US2023/012348 filed on Feb. 3, 2023 claims priority to U.S. Provisional Application Ser. Nos. 63/400,709 filed on Aug. 24, 2022, 63/319,379 filed on Mar. 13, 2022, 63/306,896 filed on Feb. 4, 2022, and 63/306,397 filed on Feb. 3, 2022, all of which are hereby incorporated by reference in their entirety.

PCT/US2021/033007 filed on May 18, 2021 claims priority to U.S. Provisional Patent Application Ser. Nos. 63/184,656, filed on May 5, 2021, 63/171,780, filed on Apr. 7, 2021, 63/167,836, filed on Mar. 30, 2021, and 63/153,223, filed on Feb. 24, 2021, all of which are hereby incorporated by reference in their entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates in general to the field of implantable medical electrodes and neuromodulation devices, and more particularly to multi-contact helical wire structure electrodes with integrated connectors for electrical stimulation, sensing, and connection to implantable pulse generators.

### 2) Description of Related Art

Currently the state of the art includes neuromodulation devices, including electrical stimulators and electrodes for interfacing with neural tissue, which have been developed for various therapeutic applications. These devices may be used for spinal cord stimulation, dorsal root ganglion stimulation, peripheral nerve stimulation, sacral nerve stimulation, direct muscle stimulation and other applications where electrical energy is delivered to or sensed from target tissues within the body.

Implantable electrodes for neuromodulation applications are typically connected to implantable pulse generators or other electrical current or voltage generators that provide the stimulation signals. The connection between the electrode and the pulse generator is often achieved through standardized connectors, such as ring contacts that interface with ball seal connectors or equivalent connection mechanisms found in commercially available pulse generators and extension cables.

Helical wire structure electrodes have been developed as an alternative to traditional lead designs. These helical structures may be formed from wire rope structures comprising multiple parallel wire strands wound into a helical configuration. Such helical wire structures can provide flexibility, self-anchoring capabilities through bunching, and the ability to be delivered through minimally invasive needle-based procedures.

When multiple independent electrical contacts are desired along a single electrode structure, the design and manufacturing of such multi-contact configurations presents various considerations. The spacing between contacts, the electrical isolation between adjacent contacts, and the mechanical stability of the transitions between helical and non-helical portions of the structure may affect the performance and reliability of the electrode.

Additionally, situations arise where a previously implanted electrode may benefit from connection to an implantable pulse generator without requiring complete removal and replacement of the electrode. Methods for modifying or adding connectors to existing implanted electrodes while maintaining the position of the electrode at the target tissue site may provide advantages in certain clinical scenarios.

Delivery systems for electrodes that include both helical and non-helical portions present design considerations, as the mechanical properties that facilitate insertion may differ between the helical and non-helical regions of the structure.

Combining helical and non-helical portions to achieve a multi-contact electrode with varying mechanical properties along the length of the electrode present additional design considerations, driven by the desired clinical delivery path and the tissue compliance and anatomical features along the delivery path.

These and other objects, features, and advantages of the present invention will become more readily apparent from the attached drawings and the detailed description of the preferred embodiments, which follow.

### SUMMARY OF THE INVENTION

Bearing in mind the problems and deficiencies of the prior art, it is therefore an object of the present invention to provide multi-contact wire structure electrode for use with neuromodulation systems.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a multi-contact wire structure electrode is provided. The multi-contact wire structure electrode comprises parallel strands of rope wire structures wound into a helical structure. The helical structure has two or more proximal stimulating electrodes and one or more distal connectors for electrical interfacing.

According to another aspect of the present disclosure, manufacturing methods are provided for integration of standard ring contacts onto a helical wire structure electrode. The helical wire structure electrode consists of one or more helical wire rope structure electrodes.

According to another aspect of the present disclosure, a device for inserting a partially helical wire structure electrode is provided. The partially helical wire structure electrode has a non-helical region for electrical connection. The device comprises an inner sheath to mechanically stabilize the non-helical region of the structure.

According to another aspect of the present disclosure, a device comprising of a combination of both helical and non-helical wire structures is provided. Alternating helical and non-helical regions along the length of the electrode results in varying mechanical properties of the lead. This linear customizable variance of mechanical properties results in an electrode that can be rigid in one desired region, such as the ring connector region, while flexible in another, such as the nerve contacting region.

According to another aspect of the present disclosure, a method is provided for adding one or more ring connectors to previously placed helical wire structure electrodes. The adding of the one or more ring connectors is performed through welding, brazing, or swaging.

The foregoing general description of the illustrative embodiments and the following detailed description thereof are merely exemplary aspects of the teachings of this disclosure and are not restrictive.

Still other objects and advantages of the invention will in part be obvious and will in part be apparent from the specification.

The above and other objects, which will be apparent to those skilled in the art, are achieved in the present invention which is directed to a multi-contact wire structure electrode, comprising:
a. a plurality of parallel strands of rope wire structures wound into a helical structure;
b. two or more proximal stimulating electrodes formed by uncoated regions of the rope wire structures; and
c. one or more distal connectors for electrical interfacing, wherein the distal connectors are configured for connection to an implantable pulse generator.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1A illustrates wire rope structures with intermittent coating in a parallel arrangement, according to aspects of the present disclosure.

FIG. 1B illustrates a top view of wire rope structures with intermittent coating arranged in parallel with an offset configuration, according to aspects of the present disclosure.

FIG. 1C illustrates a side view of a helical wire structure formed by winding the wire rope structures of FIG. 1B, according to aspects of the present disclosure.

FIG. 2A illustrates a rope wire structure in an unfolded configuration prior to being wound into a helical structure, according to an embodiment.

FIG. 2B illustrates a side view of the rope wire structure of FIG. 2A in a looped configuration, according to an embodiment.

FIG. 2C illustrates a side view of a helical wire loopback structure electrode in a wound configuration, according to an embodiment.

FIG. 3 illustrates a side view of a helical wire rope structure with two parallel wire rope structures partially wound together, according to aspects of the present disclosure.

FIG. 4 illustrates an image of a device manufactured consistent with the illustration in FIG. 3, according to an embodiment.

FIG. 5 illustrates a side view of a delivery system configured to deploy a partially helical wire structure, according to aspects of the present disclosure.

FIG. 6 illustrates a multi-contact helical wire structure with integrated ring connectors, according to an embodiment.

FIG. 7 illustrates a magnified view of a distal end of a multi-contact electrode with ring contacts, according to aspects of the present disclosure.

FIG. 8 illustrates a perspective view of a multi-contact electrode connected to an implantable pulse generator, according to an embodiment.

FIG. 9 illustrates a method for adding a connector to a previously implanted helical wire structure electrode, according to aspects of the present disclosure.

FIG. 10 illustrates a kit for inserting the electrode of the present disclosure.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As used herein, “substantially linear” means either straight, somewhat wavy or gently meandering, but not a sharp bend, corner or a bunching.

As used herein, “strand” means a length of metal wire (or other material) which may be separate from all other strands or may be part of a continuous length. If a continuous length, the ends of the wire rope and the resulting helix have no sharp ends. A continuous length must break in at least two nearby places before pieces of wire are at risk of being left inside the body if the overall structure is removed. Breaking in two places near enough for a piece to be severed from the loop or, if severed, to not be brought along during removal by being wedged into the helical structure.

As used herein, “self-bending” includes that the helical wire structure has sufficient flexibility, when it meets mechanical resistance, to bend in any direction and up to 180 degrees on its longitudinal axis, without a healthcare professional using a specific tool to bend it.

As used herein, “self-anchoring” means that the electrode needs neither sutures or other traditional anchoring techniques nor an open cut-down, or even laparoscopy, to secure the helix.

As this invention is susceptible to embodiments of many different forms, it is intended that the present disclosure be considered as an example of the principles of the invention and not intended to limit the invention to the specific embodiments shown and described.

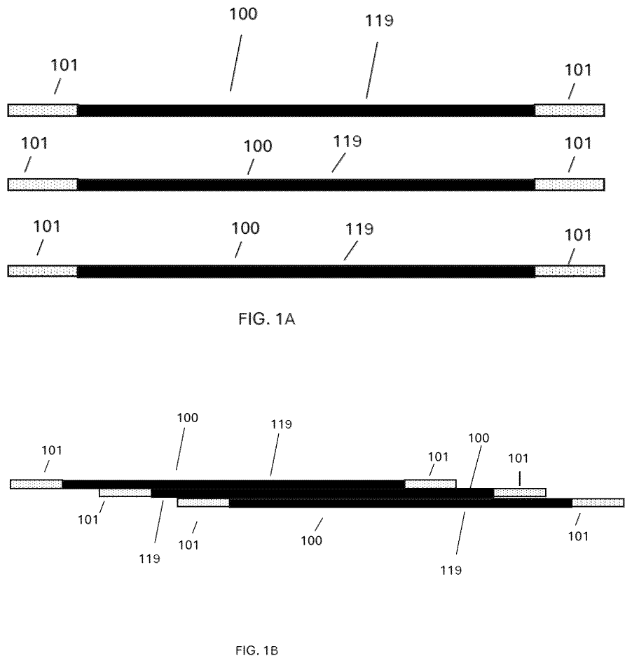

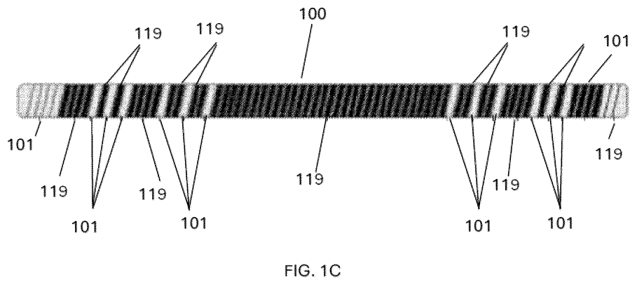

FIG. 1A, FIG. 1B and FIG. 1C shows an embodiment where three wire rope structures 100 with intermittent coating 119 FIG. 1A having proximal electrodes 101 uncoated on each end and an uncoated offset region 102 near the middle such that when placed in parallel with an offset as shown in FIG. 1B may be wound together as shown in FIG. 1C to achieve a helical wire structure 100 with three independent contacts or electrodes 101 at a spacing dependent on the number of parallel wire rope structures with intermittent coating 119, the offset between the three parallel wire rope structures, and finally the pitch and core diameters of the helical structure achieved through winding together of the wire rope structures.

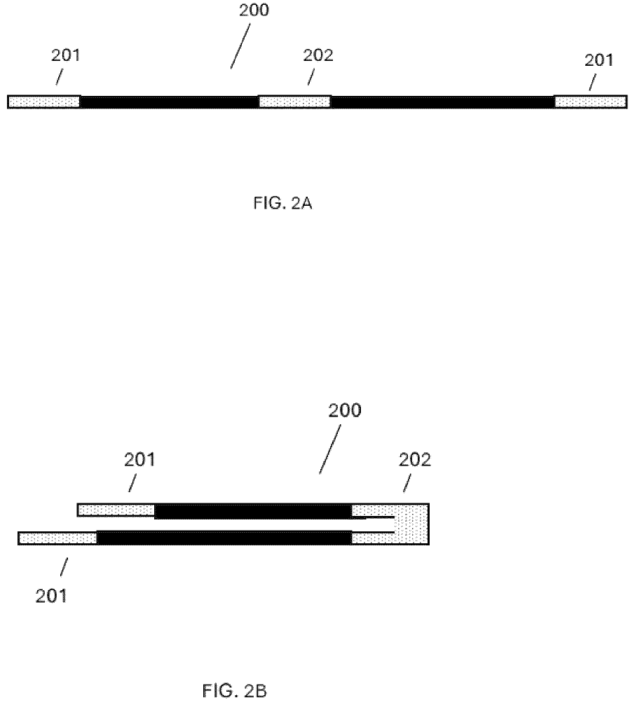

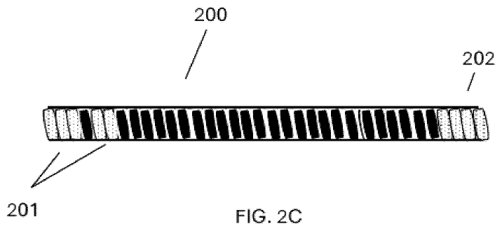

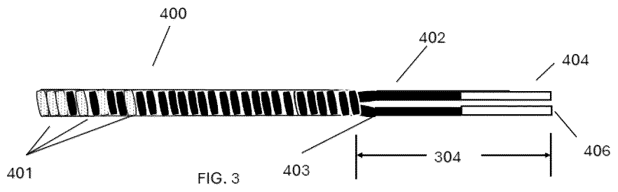

FIG. 2A, FIG. 2B and FIG. 2. C show a rope wire structure 200 with proximal electrodes 201 uncoated on each end and an uncoated offset region forming distal electrode 202 near the middle. As shown in FIG. 2B the rope wire structure is looped back upon itself with offset proximal electrodes 201 and uncoated offset region forming distal electrode 202. As shown in FIG. 2C the loopback rope wire structure is wound on a mandrel to create the helical wire loopback structure electrode 200 with two stimulating proximal electrodes 201 and one distal electrode 202 to serve as the collector.

FIG. 3 shows a helical wire rope structure 400 consisting of two parallel wire rope structures 402 and 403 that are only partially wound together as to leave an unwound section differing from the embodiment in FIG. 1 where all wire rope structures in parallel are wound together from end to end. Each non-helical ends 404 and 406 of the wire structure 400 only connects to a single uncoated electrode at the proximal end of the wire structure 400 and have electrodes 401 at the distal end and the non-helical ends 404 and 406.

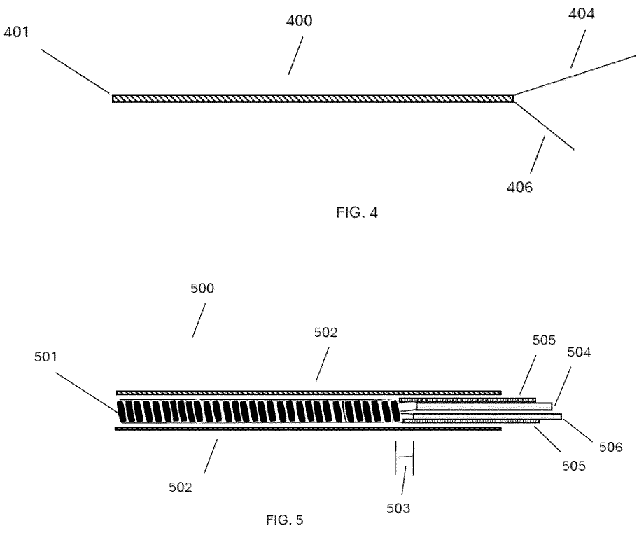

FIG. 4 shows an image of a device manufactured consistent with the illustration in FIG. 3. Where in electrodes 401 are at the distal end and non-helical ends 404 and 406 are at the proximal end of the wire structure 400.

FIG. 5 shows an embodiment of the delivery system design that would be used to deploy the partially helical wire structure 500 in FIGS. 3 and 4, where and outer cannula or needle 502 contains the partially helical wire structures 504 and 506 with one or more contacts 501 with an additional inner sleeve 505 containing the non-helical portion of the device that only extends to the transition region 503 of helical to non-helical structure. Upon deployment, the inner sleeve 505 enables pushability for insertion into a patient of the entire structure that is normally only inherent to the helical wire structure.

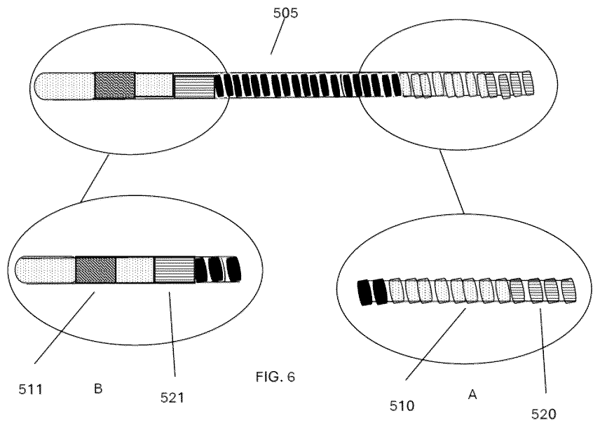

FIG. 6 shows a multi-contact helical wire structure 505 (two contacts in this specific example, while a multitude of contacts of 3, 4, 5, 6, 7, 8, 9, 10, 11, or 12 are also possible in this configuration) where the helical structure extends to ring 510 and 520 commonly found in spinal and peripheral nerve stimulators to allow for connection with a pulse generator, other electronic device or implanted pulse generators or their extension cables through standard ball seal or equivalent connectors. Detail A highlights the two electrode 510 and 520 ends and Detail B shows the standard ring connectors 511 and 521 to which the wire ropes of the helical wire rope structure 505 are electrically and mechanically connected to achieve Contact 510 and 520 associated only with Electrode 511 and 521.

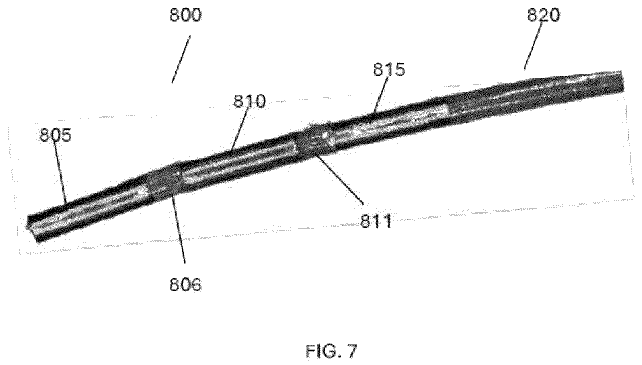

FIG. 7 shows a magnified view of a 3 ring contact distal end of the wire structure 800, with a semi-clear plastic embedding or injection molding 806 and 811 that separates the ring contacts 805, 810 and 815, stabilize physically the wire rope structures 820 as they transition from helical to non-helical in order to allow for physical and electrical connections with the individual ring contacts.

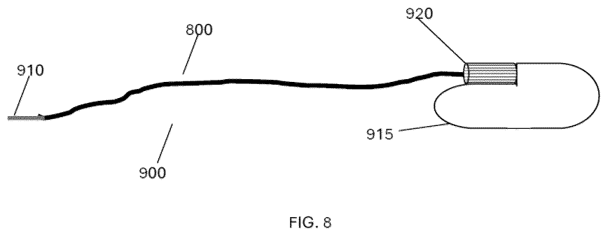

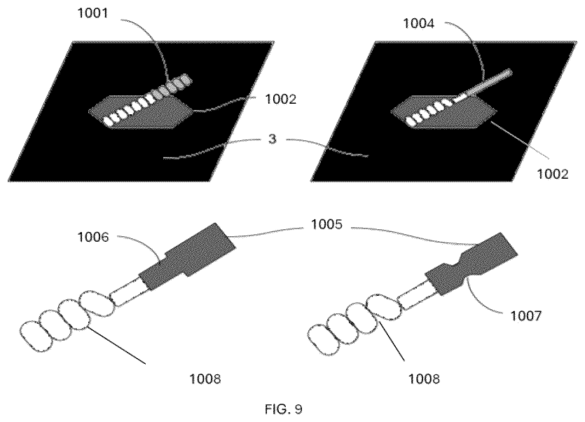

FIG. 8 shows the multi-contact electrode 800 from FIG. 7 connected to an implantable pulse generator 915. multi-contact electrode 800 has electrodes 901 and the contacts electrically attached to implantable pulse generator 915 at connector 920.

FIG. 9 shows a previously implanted helical wire structure 1001 that is partially removed through an incision 1002 in the skin 1003 and elongated contact 104 at the collector site. The cylindrical conductive 1005 contact is crimped 1006 onto the exposed and cut collector 1008. A cylindrical conductive contact is swaged 1007 onto the exposed and cut collector of the helical wire structure electrode 1001.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

One embodiment produces a helical wire structure electrode with two exposed electrodes at the proximal end placed on the electrical stimulation target (stimulators) and one exposed electrode at the distal location near or under the skin or left percutaneously (collector). The helical wire structure is fabricated from a single wire rope structure with exposed (uncoated) ends that form the proximal electrodes and an exposed (uncoated) region offset from the center of the wire rope structure that forms the distal electrode. The wire rope structure is then folded back at the distal electrode near the middle of the rope to produce two parallel ropes with the proximal electrodes offset from each other so as to not directly overlap. The resulting U-shaped parallel rope wire structure is then wound on a core to produce a helical wire structure electrode with a loopback design.

Two or more helical wire loopback structures may be combined by offsetting the loopback rope wire structures described above at a predetermined distance, placing them in parallel, and then winding the parallel structure around a mandrel.

Adding a connector to a previously implanted electrode without complete removal may be achieved in a previously placed helical wire structure electrode to connect said electrode to a pulse generator, other electronic device or an implantable pulse generator. In order to achieve this electrical connection intraoperatively, a cylindrical conductive connector may be applied to the implanted helical wire structure electrode without removal of said electrode fully from the body with the intention to maintain the proximal end of the electrode unmoved. The implanted helical wire structure electrode would have the distal component under the skin partially extracted to extend the structure back to its non-helical rope wire structure. The partially elongated electrode end that is now in a rope wire structure configuration may be cut to length after which a cylindrical contact is applied by threading the wire rope structure into the cylinder. Applying a crimping force or distance, as well as a predetermined swaging diameter reduction, may then be applied to the cylindrical contact to mechanically and electrically secure the cylinder to the electrode. In this approach, the helical wire structure electrode is converted without complete removal to an electrode that can be placed into the socket connector of an implantable pulse generator. The cylindrical contact may also be applied without partially elongating the electrode, which will require a different crimping force or depth or a different swaging diameter to achieve a reliable electro-mechanical connection.

The invention as described a helical wire structure with an integrated single pin cylindrical contact to be placed inside the socket connector of an electrical stimulator. In this embodiment multiple (2 or more) cylindrical connectors are included on the distal end of a multi-contact helical wire structure electrode. The cylindrical conductive contacts are separated by an insulative material from each other. Individual conductors of the multiple contact helical wire structure electrode are connected to each individual cylindrical contact either through welding, brazing, or using a conductive adhesive. The insulative material may partially extend into the remainder of the helical wire structure. This multi connector embodiment may be used for electrical stimulation, or other applications such as radiofrequency ablation.

The instant invention provides the healthcare professional the ability to insert the electrode rapidly without surgery and supervision from support personnel such as manufactures representatives or medical support staff.

The instant invention promotes reducing the time needed to insert the electrode and reduces the clinical logistics needed to complete the process or procedure. The process is less invasive and reduces complications. The process does not require sutures. The time of deployment is between 5 and 30 minutes wherein the preferred time is 15 minutes. The less intervention required promotes greater compliance and a shorter learning curve for the healthcare professional.

The device of the instant invention is self-anchoring and if inserted into the body the proximal end does not stick out making it less obtrusive to the patient, but may be left percutaneous for a non-chronic duration (less than one year) to achieve a direct electrical connection.

The device of the instant invention includes both helical coils and non-helical coils and structures, resulting in variable mechanical properties along the linear length of the structure

The injectable electrode in the present application presents significant improvement over prior electrodes in several ways including without limitation novel changes in the anchoring and removal characteristics for implants for neuromodulation including stimulation and block, and also ablation and any transmission of energy in a body.

Securely anchoring the lead at the target tissue is a major need not fully solved by prior art devices. Migration of a neuromodulation lead away from its target has been shown to be by far the leading device-related adverse event in at least two recent independent studies of spinal cord stimulation (SCS), dorsal root ganglion stimulation (DRG), occipital nerve stimulation (ONS), sacral nerve stimulation (SNS) and peripheral nerve field stimulation (PNFS). Lead migration made up 59% of device-related adverse events, and 27% of the adverse events as a whole in DRG. Sivanesan, E., et al., Retrospective analysis of complications associated with dorsal root ganglion stimulation for pain relief in the FDA MAUDE database, Reg. Anesth. Pain Med. 44(1): 100-106 (2019); Eldabe, S., et al., Complications of Spinal Cord Stimulation and Peripheral Nerve Stimulation Techniques: A Review of the Literature, Pain Med. 17(2): 325-36, (2016). In DRG, lead migration made up 59% of device-related adverse events, and 27% of the adverse events as a whole. Sivanesan, E., et al., Peripheral lead migrations have been reported at rates as high as 100% in a case series of ONS at 3 years and 60% at the end of 1 year and in 12 of 51 subjects (24%) in the ONSTIM study of ONS. Eldabe, supra at 326. SCS leads were found to migrate in different studies ranging from 13.2% to 27% of patients. Eldabe, supra at 326. SNS leads were found to have migrated in 16% of patients despite the use of tined leads. Eldabe, supra at 327.

Explant and revision are also major aspects of reported adverse events for DRG implants. Of the adverse events reported to FDA, 49% required at least one revision surgery and 16% required explant surgery. Each revision or explant surgery presents new infection risk.

Explant surgery was associated with 83% of deep infections, and revision surgery with 9%. Severe neurological complications were associated with explant or revision surgery in 1.8% and 1.6% respectively in patients with reported adverse events. Difficult removal was reported 6.2% of the explant surgeries. “Difficult lead removal often prompted purposeful lead cutting, whole lead retention, or inadvertent lead damage, which resulted in a retained lead segment within the epidural space. Similarly, damage to the sheath used for lead placement sometimes resulted in a retained segment.” Sivanesen, supra, at 6.

A recent review of medical device reports (MDRs) in SCS by the Food & Drug Administration (FDA) found a lower percentage of specific lead migrations, 7.2%, but the leading patient problem code was inadequate pain relief, 28.1%. https://www.fda.gov/medical-devices/letters-health-care-providers/conduct-trial-stimulation-period-implanting-spinal-cord-stimulator-ses-letter-health-care-providers In this regard, it is relevant that lead migration of only 1 mm away from the tissue target can require a dramatic increase in current output by the lead to stimulate or block the target as the second spatial differential equation for the activation for neural tissue describes. In such a circumstance, failure to increase the current reduces stimulation or block, but increasing the current too high can cause the patient great discomfort and lead to nonuse.

There have been various approaches toward anchoring the lead in place. The manufacturer of a DRG product recommends traditional anchoring (tines) and strain relief loops. These measures, however, require access to the site by the physician, which in turn leads to some trauma as a result of the need for space for surgical implements necessary for the suturing to be performed. “[L]ead migration remains the most common complication of spinal and peripheral nerve stimulation. Although paraesthesia coverage loss due to lead migration can be recaptured by reprogramming, the majority of the instances of major lead migration require minor reoperation to relocate the lead to its original position and most will incur the cost of a new lead.” Eldabe, supra, at 327. Each revision procedure presents new injection risk, and the direct and indirect costs of the foregoing complications can mount quickly for patients, payors and society as a whole.

A better solution for removal is greatly needed. Previous particle-based versions of an injectable electrode present challenges for removal of 100% of the material injected. Some previous wire-based injectables have been conducive to in-growth by body tissues but can allow too much in-growth, without a ready means for severing the in-growth, requires levels of force for removal leading to excessive trauma, bleeding and pain to the patient.

More particularly, disclosed herein is an electrode comprising a helical wire structure which solves several problems with the prior art including without limitation suture-less anchoring on or near a tissue target and removal, both anchoring and removal being minimally invasive.

The present invention includes a helical wire structure electrode which can be injected into a body near or on a tissue target such as a peripheral nerve, nerve ganglion or a tumor and be anchored without sutures. Although the helical wire structure electrode includes at least one wire rope as an intermediate stage, the wire rope by itself does not have the properties of the present invention.

An electrode comprising a helical wire structure solves the very significant concerns with the prior art in a number of ways. The invention comprises a primary wire rope comprising multiple parallel wire strands, with the wire rope being wrapped helically around a core to form a secondary wire structure, i.e., a helix, comprising coils, which is flexible, bendable, stretchable, compressible and pushable.

As used herein, “strand” means a length of metal wire (or other material) which may be separate from all other strands or may be part of a continuous length. If a continuous length, the ends of the wire rope and the resulting helix have no sharp ends. A continuous length must break in at least two nearby places before pieces of wire are at risk of being left inside the body if the overall structure is removed. Breaking in two places near enough for a piece to be severed from the loop or, if severed, to not be brought along during removal by being wedged into the helical structure.

As used herein, “dispenser” or “delivery system” means any one of a needle, cannula, catheter, tube, insert.

As used herein, “self-bending” includes that the helical wire structure has sufficient flexibility, when it meets mechanical resistance, to bend in any direction and up to 180 degrees on its longitudinal axis, without a healthcare professional using a specific tool to bend it.

As used herein, “self-anchoring” means that the electrode needs neither sutures or other traditional anchoring techniques nor an open cut-down, or even laparoscopy, to secure the helix.

For a helical wire structure comprising individual lengths of wire twisted together, the wire ends may be crimped so that they do not irritate surrounding tissue, or they may be gathered and coated in a polymer on the circumference of the end but leaving the wire ends exposed to conduct energy. Individual wire ends may also be heated so they form a mass.

The electrode herein possesses a general shape and pattern of a helix made of at least one wire rope which comprises a number of strands. Although the general shape and pattern of the helix is predictable among all embodiments given standardized manufacturing techniques, the placement of an individual strand and the actual shape and dimension of a single coil in the helix may be random or irregular, but only in this sense within a general shape or pattern. In the same way, a bunching anchor from a helical structure with a given wire composition and standardized manufacturing may be random or irregular, but only in this sense within a general shape and pattern, as one investigates the formation it becomes clear that the electrode adheres to a formal design.

The helical wire structure electrode may comprise gold wire as well as other conductive wires selected from the group consisting of gold, silver, platinum, stainless steel, titanium, titanium-nickel, iridium, platinum-iridium, tungsten, platinum-tungsten and other metal alloys such as MP35N, a cobalt-nickel-chromium alloy with molybdenum added for corrosion resistance. Wires comprising the above metals are readily available commercially in the 2-300 micron diameter range, and wires of other diameters are also suitable for some embodiments of the present invention. The final selection of material and diameter for a particular embodiment is dependent on patient biocompatibility and desired tensile (mechanical) and electrical properties for the particular application and embodiment, as well as dependent on optimum force supplied by the wire structure electrode onto the tissues against which it is pressed, because mechanical forces from any implanted electrode influence formation of encapsulation tissue. Wires of these biocompatible metals have different mechanical and electrical properties, such as conductivity, and the potential effects for heating of the wires during the conduction of electrical current. The metal composition of the wires can be varied to introduce desired physical properties. The deployment process partially unwinds some of the strands 4 from the main helical structure and pushes them between 1 to 200 microns away that is mechanically distant enough from the helical structure 1 to be mechanically free to move with the tissue and have less encapsulation between that strand and the native tissue. Also, the strands which partially unwind are very flexible (because they are so thin and can move even more easily than a coil of the helical structure) and can be closer to the tissue target and help with conduction of energy. In this way the deployment process partially unfolds the tightly wound wire rope 22 forming the helical structure and helps to create more flexibility and thus better mechanical matching between the helix and the surrounding tissue. This is a helical macrostructure with a multi-strand microstructure.

As shown in FIG. 1, FIG. 2, and FIG. 6, for example, the strands on the outside of the helical wire structure allow for a soft integration with the surrounding tissues allowing for a pliable interfacing of the overall structure with the tissue. Also, without a guidewire, because the inner diameter of the helical wire structure defines a core that is hollow and, to the extent the individual coils are not bonded together, the coils can move in relation to one another by bending, stretching and compressing. The ability to “give” when the body moves reduces compression and inflammation of the tissue, resulting in a thinner encapsulation than for traditional neuromodulation devices with a single wire or a metal plate or large volume metal surface pressing against tissue. A thinner encapsulation response affords better electrical coupling (lower impedance) interfacing. The body's encapsulation of the invention herein is less than 100 microns, compared to encapsulation of prior art devices in a range up to 1000 microns. The stiffness of prior art electrodes, which are stiff in all dimensions, is a significant contributor to generating a thick capsule around it. The thicker the capsule, the less energy can pass through it.

The helical structure allows for in-growth of tissue including collagen and vessels to partially penetrate between strands, anchoring the coil without the need to suture the overall structure down to the target tissue. Natural integration with the surrounding tissue aids the overall capability of minimally invasive delivery via a thin (e.g., 18 gauge) needle because no sutures are needed. The mechanical integration caused by in-growth of tissues and the encapsulation of the electrode results in anchoring to the outer surface of the structure. The wire rope itself is not nearly as penetrable by tissue in-growth as is the helical structure which allows movement between coils and has a hollow core.

The helical structure is highly flexible, configured to fold along its longitudinal axis by 10 degrees, 30 degrees, or even 180 degrees in any direction (that is, back on its own original axis) as measured from its longitudinal axis. As the healthcare professional is viewing the dispenser tip arrive at the tissue target (e.g., through fluoroscopy or ultrasound), healthcare professional may hold the tip substantially stationary and begin pushing the electrode from the tip. The surrounding tissue resists the emerging electrode and deflects its path so that its flexibility causes it to bunch near the tip. Bunching during injection is an intentional characteristic designed to allow the healthcare professional to optimally fill a void (either created or naturally occurring) inside the body, either inside or near to the target tissue or structure of interest. The bunched macro-structure of meandering twists and loops creates compression against the tissue, or fills a created void, so that the bunching is larger in diameter than the outer diameter of the dispenser which has generated an insertion channel. That is, the deposit of a width of bunched coils of the helical wire structure wider than the dispenser and insertion channel, creates a mechanical impediment which keeps it in place during normal bodily movements.

Bunching of the helical wire structure allows for the custom design of the electrode by the healthcare professional using an electrically insulated dispenser while in electrical contact with the target tissue during the insertion. This includes a smaller or larger bunching anchor for mechanical anchoring but also more or less electrically active surface area on or near a tissue target. The bunching anchor is enlarged or reduced in size during insertion from an electrically insulated dispenser by pushing it out and retracting it back into the needle and using the dispenser to provide insulation during the testing of stimulation, block, ablation or similar with the insulated/insulating needle still present. This provides the healthcare professional with a very versatile tool while visualizing the injection dispenser itself as a part of the stimulation, block, ablation or similar system and let the bunching anchor form in front of the tip of the dispenser. Once the customized shape and size has been deployed and electrically verified to be optimal, insulator or mechanical glue may be deployed from the dispenser to insulate around the substantially linear portion of the helical wire structure leaving only the bunching anchor to electrically interface with the tissue.

As used herein, “substantially linear” means either straight, somewhat wavy or gently meandering, but not a sharp bend, corner or a bunching.

Bunching of the helical wire structure prevents pistoning, a phenomenon in which a round or smooth structure, say a traditional or conventional lead wire, is able to move back and forth in a fibrous capsule. Bunching anchors, as described herein, may have a general shape which is consistent with the composition of the wire rope and the type of void, but bunching anchors are not a single, exact shape. Rather the exact shape of a specific bunching anchor results from several factors including the diameter(s), number and material of the strands, the tightness of the twisting of the wire rope, and the particular shape and character of the void into which they are injected or creates by displacing tissue. All of these properties of bunching anchors, and the helical macrostructure with a multi-strand microstructure (discussed herein elsewhere), contribute to resistance to pistoning.

The bunching anchor may be achieved in the helical regions of all embodiments described herein, and is thus configured to help create a void and also to take on the shape of an existing void by mechanically interacting with the tissue in which it is deployed. It may retain that shape by forming a negative imprint of the shape of the interfacing tissue, so that it assumes complex shapes inside or near the target tissue, partially or fully surrounding the target tissue for an optimal mechanical and electrical interfacing with a target tissue. It can fill a space inside a mechanically harder tissue (e.g., bone), a canal inside a bone, other more resilient body tissue if this void is being created during the deployment process within a naturally occurring soft tissue filled volume inside the mechanically harder tissue. An example is a channel passing through a bone that is filled with nerve and fatty tissue. Another example is a nerve entering an organ, i.e., the innervation point of an organ, where a void may be formed just beneath the endothelial tissue surrounding the organ, thus allowing for the mechanical fixation of the helical wire structure inside the organ right beneath the endothelial tissue and near an innervation point. This helical wire structure can also fill a void within a sheath surrounding a nerve or other tissue target like an organ, allowing for the anchoring of the helical wire structure inside the target's sheath without the need to suture the helical wire structure to the nerve.

The helical wire structure's flexibility allows for a deployment by a bent or curved dispenser into a shape that is not a straight line. The deployment into a non-straight line allows it to provide an energy concentration effect (“lens effect”) of energy delivered by the wire structure, thereby concentrating delivered energy into a target tissue.

As implanted, the helical wire structure electrodes cross tissue boundaries and resist shear forces more than non-helical structures because the former bend, stretch, compress and contract because they are hollow, helical, and comprise many wire strands which move in relation to one another. The present invention, when deployed into tissue planes, between tissues, into neural tissue or into a neural sheath, is mechanically adaptive with movement of the sheath with respect to the surrounding biological tissue. This ability to flex and bend (because of the movement of wire strands relative to one another) more readily than solid objects to resist breaking due to the inherent formation of a rope like structure, combined with the ability to fold in on itself, meander, fill an intentionally created void that is being filled in part with a meandering and folding helix to form an anchor inside a nerve sheath or at a neural innervation point to an organ allows for the creation of a neural interface by needle without the need to suture down the folded wire structure, thus a significantly less invasive delivery procedure, a significant advantage for the patient, the physician (time) and clinic (time per patient) and payer (cost per procedure).

The bending and meandering of the helical structure is visible on ultrasound and fluoroscopy, aiding the healthcare professional with the ability to visualize the structure they are creating during the deployment process non-invasively.

The helical wire structure may be formed into a helix with a trailing (or front-running) tail of rope that is not fully wound into the helix.

Instead of a single wire, a drawn filled tube (DFT) may be used to produce the initial spool of thin wire that will then be used to manufacture the parallel wire strands. The advantage of using DFTs is that they may have an outer interface metal (e.g., platinum) and an inner more electrically consecutive metal such as silver or gold to aid with the minimization of impedance across the long distance of the parallel wire strands inside the entire length of the rope making up the helix of the helical wire structure electrode.

Different densities and volumes of wire deployed at different locations throughout the cavity provide for different mechanical properties (higher stiffness of the final macro structure, or higher flexibility of the macro structure where needed) as well as different electrical properties (higher electrode to electrolyte interface area to allow more charge to be deployed at specific locations) as needed.

The many wire strands in the wire rope ensure that electrical conductivity is maintained even if one or a group of the single wire strands were to be severed during the manufacturing process, the storage, the shipping, the deployment into the living body or during the duration of being located inside the body and potentially being subjected to stretch and shear forces within the body. In the example of 100 parallel strands of gold wire, losing the continuous connection of up to 50 of the wires would only slightly increase the overall impedance of the entire helical wire structure—but the severing of up to 50 of the strands is highly unlikely without being cut by a tool. The twisting of the parallel wire strands prior to helical winding ensures that there is a tight packaging of the overall wire per helical structure volume. Furthermore, if one or more of the wires were to be interrupted at specific locations then they remain attached to the entire structure, reducing the possibility of loose particles being separated from the structure as a single strand would need to break in two nearby locations to create one loose piece. A single break would not create a loose piece of strand, just two open ends which by their own nature are then even less likely to break off due to increased mechanical flexibility of the open ends when compared to the relatively higher stiffness of a wire loop prior to the loop breaking. If the rope were not composed of many (i.e., 5, 10, 50, 100, 200 or more) strands, but only one strand, then mechanical bending and shear forces would be more likely to cause a fatigue break that may result in loose pieces. The creation of the wire rope from numerous strands greatly reduces the risk of a fatigue break creating loose pieces, increasing the probability that the entire structure will be removed as a whole if undertaken.

The following is one embodiment of a manufacturing method for the helical wire structure:
1. Wind metal wire at a set distance around 2 or more mandrels to form 2 to 20,000 parallel strands.
2. Optional: add additional mandrels for skeining wire or other material of different diameter.
3. Remove parallel strands from mandrels and twist along the longitudinal axis to form a rope.
4. Optional step: apply mechanical stabilizer or insulator to rope at specific locations (such as a glue) and let it dry to ensure the rope does not significantly unwind during the helix winding process.
5. Optional step: connect ring contacts to each of the wire rope structures and embed one or more ring contacts in a concentric linear fashion in a stabilizing material such as polyurethane, polyethylene, PEEK, epoxy, or silicone
6. Wind rope around a guidewire of outer diameter (preferably within a range of 0.1 to 2.0 mm) to form a helical wire structure around the guidewire with an outer diameter of the helix structure within a range of 0.2 to 3.0 mm.
7. Optional step: apply mechanical stabilizer or insulator to the helical wire structure electrode at specific locations (such as a glue) and let dry to ensure the rope does not unwind during the helical winding process.
8. Optional step: Further encase the space around and between the ring contacts (if present) including the transition(s) from non-helical to helical structure(s).

The mandrel-based manufacturing of the wire rope leads to ends which are rounded when formed by continuous wires, thereby lessening the possible risk of sharp wire ends unfavorably damaging tissue or breaking off loose pieces. The mandrel-based manufacturing step of the initial rope leads to rope formed by 3-5 to 100 s of parallel strands of single wires which increases the reliability of the electrical conductivity of the helical wire structure. If, during the acute or chronic deployment of the helical wire structure, one or a few of the single wire strands were to break, then there is only an insignificant reduction in the overall ability of the helix to conduct electrical energy. The winding of parallel wires and helical twisting of the wire structure causes a higher compression ratio of wires to volume closer to the core of the structure, limiting the amount of cell ingrowth at the inside vs the amount of cell ingrowth and subsequent mechanical anchoring towards the outside of the wire rope comprising the larger helical structure.

The manufacturing apparatus for a wire rope of parallel wires with looped ends twisted to a precise diameter in one embodiment comprises: 1. At least two mandrels 2. Motor (with or without force measurement and/or torque measurement) 3. Velocity controller 4. Visual or electrical or mechanical sensors to detect continuity of the supplied wire 5. Wire Tensioner (with or without pins, guides, or clamps) 6. Wire Twisting Motor

The manufacturing apparatus for producing a helical wire structure electrode from at least one wire rope comprises a Guide Wire tensioner and holder: 1. Rope wire structure securing mechanism 2. Winding motor 3. Rope tensioning sensor and controller during winding

The product remains on the guide wire for transfer to a delivery cannula or dispenser.

Cleaning of the helical wire structure electrode occurs during several steps throughout the manufacturing process as well as within the supply chain. Components with direct human contact, at a minimum, undergo a cleaning operation prior to assembly into the final medical device. Manufacturing aids that are in direct contact with these components (such as mandrels and guides) are also cleaned on a regular basis to prevent transfer of any foreign materials to the finished device during manufacturing. Once wound into the helical wired electrode structure, the product undergoes another cleaning operation. Items used in the cleaning process include without limitation ultrasonic cleaning baths, isopropyl alcohol, water soluble detergent and mechanical cleaning apparatuses (e.g., brushes, wipes). Manufacturing of the helical wired electrode and delivery system occurs in an environmentally controlled area. Personnel are required to wear PPE (personal protective equipment) such as gloves and lab coats to prevent the introduction of foreign materials into the device or components. One of the benefits to the helical/mesh shape and structure in a cleaning operation is that it provides maximum surface area for cleaning agents and methods to contact the electrode effectively. The cleaning process for the helical wired electrode is detailed and recorded in manufacturing documentation and has been validated.

Ability to remove the structure is aided by the mechanical stability of the structure and tear resistance based on the folding structure, size of core wire and number of wires used.

Ease of removal of the helical wire structure is one of its distinct advantages. After applying local anesthetic, the physician makes a small incision on the skin above the uppermost portion of the electrode and pulls out the helical wire structure which unzips individual coils of the structure as they release from the tissue first pulling inward towards the center line of the helical wire structure electrode before being pulled outward to exit the body. When a coil 506 is pulled, the outer diameter contracts and pulls the outer surface away from surrounding tissue. This releases the inner encapsulation layers from the outer encapsulation layers one coil at a time, allowing the removal at low overall forces as one loop separates tissues mechanically prior to moving outward instead of prior electrodes and leads that require the attached encapsulation layers to remove along the entire implant at once. The separation from encapsulation lays one coil after the other ensures low removal forces and greatly adds to the reliability of the removal process that is easily performed in a clinic or outpatient procedure without major risk of the helical wire structure electrode rupturing, breaking (or “cutting” as healthcare professionals refer to it) of a lead or electrode structure. The in-built quality to deform one coil at a time and thereby reduce the outer diameter of the helical wire structure electrode, while stretching the total length of the helical wire structure, swaps larger outer diameter for more length during the removal process, thereby reducing the removal forces, similar to how an inclined plane reduces the forces needed to lift an object one small incremental step at a time instead of lifting it all at once. This process of incremental removal of one coil at a time, herein described as “unzipping,” is of benefit to the healthcare professional conducting the removal process (and the patient) as unzipping reduces the possibility of loose pieces, aids with the release from the encapsulation tissue as well as delicate native tissues and enables exiting the body via its own path or footprint occupied chronically inside the body prior to removal.

In other embodiments, the helical wire structure may be the front end of a system that incorporates the front end for bunching and followed by an insulated lead portion that does not have the ability to fold when placed into tissue, potentially followed by a connector or an interfacing unit to be able to connect to another foldable structure, interconnecting structure or implantable pulse form generator. Such a system uses the helical wire structure to provide mechanical anchoring forces that are established during the placement procedure and allow for the entire system to be left inside the body without the need to be mechanically anchored further with the aid of sutures or other traditional techniques.

There may be locations of higher stiffness (e.g., via added glue, heightened tightening via rolling or other means of mechanical stiffening) of the manufactured wire structure along its longitudinal axis and there may be locations of lower stiffness (e.g. by lesser rolling or lesser compression during the out-of-body manufacturing process) to allow for predetermined locations of folding during the deployment process. An example is for the helical wire structure to be stiff for 2 mm in length followed by 1 mm in lesser mechanical stiffness. Another example is for the manufactured helical wire structure to be stiff for more than 2 mm in length (such as 5 mm or 10 mm or 20 mm) followed by 0.5 mm (or more or less than 1 mm) in lesser mechanical stiffness. Such a helical wire structure with the example of a 2 mm which is stiffer and 1 mm which is less stiff lengths along its longitudinal axis is be able to create a helical wire structure electrode of meandering waves of approximately 1 mm amplitude waves height (half the 2 mm stiff length) during the placement process.

During injection, the helical wire structure may be pushed into and retracted (fully or partially) into the cavity to enable creation and filling of the cavity within or adjacent to the target tissue. The helical wire structure may also expand the cavity crated during the injection.

After the electrode has been placed, the wound (e.g., needle puncture) may be closed with sutures, steri-strips or suture glue.

Following integration by the body's encapsulation reaction to the electrode, the helical wire structure may be used to transfer energy to or from the target tissue inside the body to another location within the body, such as a location closer to the skin (i.e., a subcutaneous pocket at a depth of about 1 to 3 mm inside the body).

The electrode may be used to transfer electrical, thermal, acoustic, mechanical or electrical energy to initiate a dislodging process from the tissue surrounding the electrode.

In certain instances, the helical wire structure may be used to supply electrical stimulation or blocking energy to neural tissue, or longer-term electrical blocking energy that induces the changes of the pH in the direct vicinity of the electrode, such as placed in close proximity to neural tissue to allow for an electrically induced chemical lesion (injury/dissolution) of a tissue target, or electrical current to induce a change in pH in the direct vicinity of some or all of the wires of the helical wire structure electrode to induce cell death of the first few cell layers mechanically anchoring the electrode. This chemical lesioning may be used minutes, hours or days preceding a minimally invasive removal procedure. The electrode herein is highly conductive for electric current but also other forms of energy including radiofrequency current, microwave current and direct current, as well as ultrasound.

The electrode may be optimized for a minimally invasive removal days, weeks or months after the placement procedure. Such a removal may be achieved by pulling, twisting, turning or otherwise mechanically engaging with the electrode.

The present invention may be used as a standalone device or may be connected to a conventional lead which, in the prior art, is a single or group of wires insulated via a weld and or adhesive, or the connection may be established mechanically and stabilized via an adhesive. The adhesive may be non-conductive to provide electrical shielding and limit galvanic interactions between the wire structure and lead wire materials, or it may be conductive to ensure a more robust electrical connection. A conventional lead may have a helical wire structure connected on each end, with needle injection devices of different lengths. One side may be injected to a target, with the other injected subcutaneously.

During the stranding and winding process, or thereafter, a conventional lead may be indirectly integrated to the helical wire structure or partial coating applied to the wire structure to form lead segments for a directly integrated lead. In the mono-polar setup, this lead integrated wire structure connects an anatomical target to some distal point. In the bi-and multi-polar setup, this lead integrated wire structure connects multiple target points to multiple distal points in a device.

In other embodiments the helical wire structure electrode also comprises an integrated conventional lead. The lead may be directly or indirectly integrated, as achieved through a combination of fabrication methods. Integration of the lead alters the cellular ingrowth and mechanical integration within desired regions, while providing electrical connections between wire structures located at different anatomical targets. The lead integrated wire structure has mono-polar and multi-polar design configurations, allowing for interfacing with successive anatomic targets within one injection window and procedure. Delivery of the lead integrated wire structure is achieved either through standard dispenser channel methods commonly used to place leads, or may be achieved through front-loading of the interfacing bare wire structure regions only that do not comprise the lead region, which is placed in parallel to the wire structure delivery cannula. The latter approach simplifies the delivery mechanism to accommodate an overall shorter, and therefore more pushable, wire structure. This wire structure may also be delivered at incident angles and in complex curvatures using curved or bendable delivery needle systems.

As described herein, the helical wire structure comprises multiple stranded wires, are made of an inert conductor such as platinum or gold, and may be partially coated with inert materials such as Parylene C, Fluorinated polymers, or Silicones to form “lead” regions. Coating schemas of the wires will modulate cellular ingress rate and ability, with effects on mechanical properties of the device and required removal forces. Coating may be achieved either by micro coating of individual wires versus macro coating of multiple wires. Coating of individual wires with <15 micron (especially <5 micron) coatings will maintain mechanical flexibility and biological integration of the mesh structure to mechanically anchor the device. Coating of multiple wires, such as conventional leads, are not readily integrated and provide a smooth/slippery biological conduit that allows for free movement of the lead. Inert coatings, dependent on thickness, also provides electrical or thermal insulation from surrounding tissue at both the micro or the macro scale.

There can be indirect integration of a lead wire, optionally that may be coated with another material or mechanically achieved with the wire structure through interweaving and twisting of the lead materials' conductive wire element and the wires of the wire structure. The indirect integration step may occur during or at the end of the fabrication of the wire structure. A mechanical connection alone is sufficient. Indirect integration of a lead wire to a wire structure is also achieved through the use of welding or brazing approaches. Following welding, brazing, or crimping, the connected surface may be treated and coated as necessary

There can also be direct lead integration into a wire structure with partial coating of the wire structure at intermediate stages of the fabrication process. Coatings may be achieved through dip-spin coating of polymers or via vapor deposition methods. Coatings must be flexible to withstand intended torsion and folding of the wire structure during placement and use. The surface of the wire structure may be treated to improve adherence and allow for improved structural integrity of applied partial coating.

In other embodiments two or more wire structures with integrated leads may be combined into one wound helical wire structure that has multiple staggered bare wire structure regions that allows for successive electrical connections isolated from each other into one wire structure. The winding pattern may be regular with equally spaced regions between each wire structure in the multi-polar parallel pre-stranding to post winding product, or may be intermittently altered where one structure may traverse several other structures over the course of single winding.

Delivery of the wire structure, with or without integrated ring connectors, may be achieved through curved needles or flexible cannulas. Potential targets that are amenable to delivery at incident angles or with complex curvature include but are not limited to the following: nerves, externally or into the sheath; anatomical targets located behind sensitive tissues (e.g., blood vessels, nerve plexus or ganglia) or bony structures; between muscle planes; and into or around tumors

Embodiments include straight needles with slanted/notched tips, straight needles with side ports, straight needles with side ports and inside flexible tubes intended to exit at a 10 to 90 degree angle from the side port with the helical wire structure electrode being able to then exit from the bent tube, flexible actuating needles, or curved needles with front or side ports.

Prior to a discussion of the preferred embodiment of the invention, it should be understood that while the features and advantages of the invention are illustrated in terms of a multi-contact wire structure electrode.

The following description sets forth exemplary aspects of the present disclosure. It should be recognized, however, that such description is not intended as a limitation on the scope of the present disclosure. Rather, the description also encompasses combinations and modifications to those exemplary aspects described herein.

Multi-contact helical wire structure electrodes comprise parallel strands of rope wire structures wound into a helical configuration. Such electrodes provide electrical stimulation, sensing, and connection capabilities for interfacing with implantable pulse generators. The helical configuration enables the electrode to be flexible, bendable, stretchable, compressible, and pushable, allowing for minimally invasive delivery and self-anchoring within tissue without sutures.

Referring to FIGS. 1A, 1B, 1C, 2A, 2B, 2C, 3, 4, 5, 6, 7, 8, and 9, various embodiments of multi-contact helical wire structure electrodes and associated delivery systems and connectors are illustrated. A helical wire structure 100 comprises wire rope structures with an intermittent coating 119 and proximal electrodes 101 at uncoated regions. A helical wire loopback structure electrode 200 includes proximal electrodes 201 and a distal electrode 202. A wire structure 400 comprises a first parallel wire rope structure 402 and a second parallel wire rope structure 403 with electrodes 401, non-helical ends 404 and 406, and an unwound section 304. A partially helical wire structure 500 includes contacts 501, an outer cannula 502, a transition region 503, a helical wire structure 504, a multi-contact helical wire structure 505, and a non-helical wire structure 506. The multi-contact helical wire structure 505 extends to a first electrode 510 with a first ring connector 511 and a second electrode 520 with a second ring connector 521. A multi-contact electrode 800 includes a first ring contact 805, insulative embedding 806, a second ring contact 810, insulative molding 811, a third ring contact 815, and a wire rope structure 820. An electrode assembly 900 comprises a helical wire structure 910, an implantable pulse generator 915, and a connector 920. A helical wire structure 1001 is shown with skin 3, an incision 1002, an elongated contact 1004, a cylindrical conductive contact 1005, a crimp 1006, a swage 1007, and an exposed collector 1008.

The helical wire structure electrode comprises conductive wires selected from the group consisting of gold, silver, platinum, stainless steel, titanium, titanium-nickel, iridium, platinum-iridium, tungsten, platinum-tungsten, and metal alloys such as MP35N, a cobalt-nickel-chromium alloy with molybdenum added for corrosion resistance. Wires comprising these metals are commercially available in the 2-300 micron diameter range, and wires of other diameters are suitable for various embodiments. In some cases, a drawn filled tube (DFT) is used to produce the initial spool of thin wire for manufacturing the parallel wire strands, where the DFT has an outer interface metal such as platinum and an inner more electrically conductive metal such as silver or gold.

The multi-contact helical wire structure has a multitude of contacts, including configurations with 3, 4, 5, 6, 7, 8, 9, 10, 11, or 12 contacts. The helical wire structure is highly flexible and is configured to fold along its longitudinal axis by 10 degrees, 30 degrees, or 180 degrees in any direction as measured from the longitudinal axis. The body's encapsulation response to the helical wire structure electrode is less than 100 microns, compared to encapsulation of prior devices in a range up to 1000 microns.

The helical wire structure is formed into a helix with a trailing or front-running tail of rope that is not fully wound into the helix. The wire rope is formed by 3-5 to hundreds of parallel strands of single wires, which increases the reliability of the electrical conductivity of the helical wire structure. The rope is wound around a guidewire of outer diameter within a range of 0.1 to 2.0 mm to form a helical wire structure around the guidewire with an outer diameter of the helix structure within a range of 0.2 to 3.0 mm.

The time of deployment is between 5 and 30 minutes, with a deployment time of 15 minutes being suitable for many applications. The electrode is left percutaneous for a non-chronic duration of less than one year to achieve a direct electrical connection in some configurations. Delivery of the wire structure is achieved through curved needles or flexible cannulas, including straight needles with slanted or notched tips, straight needles with side ports, straight needles with side ports and inside flexible tubes intended to exit at a 10 to 90 degree angle from the side port, or curved needles with front or side ports.

The insulative material separating the cylindrical conductive contacts is achieved through semi-clear plastic embedding or injection molding that separates the ring contacts and stabilizes physically the wire rope structures as the wire rope structures transition from helical to non-helical configurations. Individual conductors of the multiple contact helical wire structure electrode are connected to each individual cylindrical contact through welding, brazing, or using a conductive adhesive. The cylindrical conductive contact is applied by threading the wire rope structure into the cylinder and then applying a crimping force or distance, as well as a predetermined swaging diameter reduction, to mechanically and electrically secure the cylinder to the electrode.

Coating of the wires is achieved through micro coating of individual wires or macro coating of multiple wires, with coating of individual wires with less than 15 micron coatings, and in some cases less than 5 micron coatings, maintaining mechanical flexibility. Coatings are achieved through dip-spin coating of polymers or via vapor deposition methods. The wires are partially coated with inert materials such as Parylene C, fluorinated polymers, or silicones to form lead regions.

Locations of higher stiffness are provided via added glue or heightened tightening via rolling or other means of mechanical stiffening along the longitudinal axis, and locations of lower stiffness are provided by lesser rolling or lesser compression to allow for predetermined locations of folding during deployment. The helical wire structure is stiff for 2 mm in length followed by 1 mm in lesser mechanical stiffness in some configurations, or stiff for more than 2 mm such as 5 mm, 10 mm, or 20 mm followed by 0.5 mm or more or less than 1 mm in lesser mechanical stiffness in other configurations.

The electrode is used to transfer electrical, thermal, acoustic, mechanical, or electrical energy to initiate a dislodging process from the tissue surrounding the electrode. The electrode is highly conductive for electric current and also for other forms of energy including radiofrequency current, microwave current, and direct current, as well as ultrasound. The multi-connector embodiment is used for electrical stimulation or other applications such as radiofrequency ablation.

The delivery system comprises an outer cannula or needle containing the partially helical wire structures with an additional inner sleeve containing the non-helical portion of the device that extends to the transition region of helical to non-helical structure. Two or more helical wire loopback structures are combined by offsetting the loopback rope wire structures at a predetermined distance, placing the loopback rope wire structures in parallel, and then winding the parallel structure around a mandrel.

The wound is closed with sutures, steri strips, or suture glue after the electrode has been placed. The dispenser is any one of a needle, cannula, catheter, tube, or insert. The manufacturing method includes winding metal wire at a set distance around 2 or more mandrels to form 2 to 20,000 parallel strands. Individual wire ends are heated so the wire ends form a mass, or the wire ends are crimped so that the wire ends do not irritate surrounding tissue, or the wire ends are gathered and coated in a polymer on the circumference of the end but leaving the wire ends exposed to conduct energy.

The helical wire structure is used to supply electrical stimulation or blocking energy to neural tissue, or longer-term electrical blocking energy that induces changes of the pH in the direct vicinity of the electrode to allow for an electrically induced chemical lesion of a tissue target. The winding pattern is regular with equally spaced regions between each wire structure in the multi-polar parallel pre-stranding to post winding product in some configurations, or is intermittently altered where one structure traverses several other structures over the course of single winding in other configurations.

The indirect integration of a lead wire to a wire structure is achieved through interweaving and twisting of the lead materials' conductive wire element and the wires of the wire structure, or through the use of welding or brazing approaches. The delivery is achieved through front-loading of the interfacing bare wire structure regions that do not comprise the lead region, which is placed in parallel to the wire structure delivery cannula. The deployment process partially unwinds some of the strands from the main helical structure and pushes the strands between 1 to 200 microns away, which is mechanically distant enough from the helical structure to be mechanically free to move with the tissue.

Referring to FIG. 1A, three wire rope structures 100 with the intermittent coating 119 are shown in a parallel arrangement. Each wire rope structure 100 includes a proximal electrode 101 that is uncoated at each end of the respective wire rope structure 100. The intermittent coating 119 is applied along portions of each wire rope structure 100, leaving the proximal electrodes 101 exposed for electrical contact. The wire rope structures 100 are depicted as elongated horizontal elements, with the proximal electrodes 101 appearing as dotted or textured sections at the left and right ends of each structure, while the intermittent coating 119 covers the central portions shown as solid regions.

With continued reference to FIG. 1A, each wire rope structure 100 is formed by 3-5 to hundreds of parallel strands of single wires, which increases the reliability of the electrical conductivity of the helical wire structure 100. The wire rope structures 100 comprise conductive wires selected from the group consisting of gold, silver, platinum, stainless steel, titanium, titanium-nickel, iridium, platinum-iridium, tungsten, platinum-tungsten, and metal alloys such as MP35N, a cobalt-nickel-chromium alloy with molybdenum added for corrosion resistance. Wires comprising these metals are commercially available in the 2-300 micron diameter range, and wires of other diameters are suitable for various embodiments. A drawn filled tube (DFT) is used to produce the initial spool of thin wire for manufacturing the parallel wire strands, where the DFT has an outer interface metal such as platinum and an inner more electrically conductive metal such as silver or gold.

Referring to FIG. 1B, a top view of the three wire rope structures 100 with the intermittent coating 119 arranged in parallel with an offset configuration is illustrated. Each wire rope structure 100 includes the proximal electrodes 101 that are uncoated regions located at each end of the respective wire rope structure 100. The three wire rope structures 100 are positioned parallel to one another with a lateral offset, such that the proximal electrodes 101 of each wire rope structure 100 do not directly overlap with the proximal electrodes 101 of adjacent wire rope structures 100. This offset arrangement of the parallel wire rope structures 100 with their respective proximal electrodes 101 and the intermittent coating 119 represents an intermediate configuration prior to winding the structures together to form a multi-contact helical wire structure electrode.

With continued reference to FIG. 1B, the manufacturing method includes winding metal wire at a set distance around 2 or more mandrels to form 2 to 20,000 parallel strands. The spacing and offset between the three parallel wire rope structures 100 influences the final electrode spacing in the wound helical configuration. The wire rope structures 100 are wound around a guidewire of outer diameter within a range of 0.1 to 2.0 mm to form the helical wire structure 100 around the guidewire with an outer diameter of the helix structure within a range of 0.2 to 3.0 mm.

Referring to FIG. 1C, a side view of the helical wire structure 100 formed by winding the multiple wire rope structures 100 together is illustrated. The helical wire structure 100 comprises a series of proximal electrodes 101 positioned at intervals along the length of the structure. Between the proximal electrodes 101, sections of the intermittent coating 119 are applied to provide electrical insulation at designated regions. The proximal electrodes 101 appear as uncoated regions that serve as independent contacts or electrodes along the helical wire structure 100.

As further shown in FIG. 1C, the spacing between the proximal electrodes 101 is determined by the number of parallel wire rope structures 100 used, the offset between the parallel wire rope structures 100, and the pitch and core diameters achieved through the winding process. The intermittent coating 119 sections alternate with the proximal electrodes 101 to create a multi-contact configuration where each proximal electrode 101 functions independently for electrical stimulation or sensing purposes. The helical wire structure 100 extends horizontally with the coiled configuration visible throughout its length, demonstrating the wound arrangement of the wire rope structures 100 that form the overall electrode assembly.

Referring to FIG. 2A, a rope wire structure is shown in an unfolded configuration prior to being wound into a helical structure. The rope wire structure is depicted as an elongated linear element with intermittent coating along its length. The rope wire structure includes a proximal electrode 201 that is an uncoated region located at each end of the structure. A distal electrode 202 is positioned as an uncoated offset region near the middle of the rope wire structure. The proximal electrodes 201 appear as dotted or textured sections at the left end and right end of the structure, while the remaining portions of the rope wire structure are shown as solid sections representing the coated regions. This configuration represents the initial state of the rope wire structure before the rope wire structure is looped back upon itself and wound on a mandrel to create the helical wire loopback structure electrode 200.

With continued reference to FIG. 2A, the rope wire structure is fabricated from a single wire rope structure with exposed uncoated ends that form the proximal electrodes 201 and an exposed uncoated region offset from the center of the wire rope structure that forms the distal electrode 202. The wire rope structure comprises conductive wires selected from the group consisting of gold, silver, platinum, stainless steel, titanium, titanium-nickel, iridium, platinum-iridium, tungsten, platinum-tungsten, and metal alloys such as MP35N. Individual wire ends are heated so the wire ends form a mass, or the wire ends are crimped so that the wire ends do not irritate surrounding tissue, or the wire ends are gathered and coated in a polymer on the circumference of the end but leaving the wire ends exposed to conduct energy.

Referring to FIG. 2B, a side view of the rope wire structure in a looped configuration forming part of the helical wire loopback structure electrode 200 is illustrated. The rope wire structure is shown folded back upon itself to create two parallel segments. The proximal electrodes 201 are visible as uncoated regions located at each end of the rope wire structure, with the two proximal electrodes 201 offset from each other so as to not directly overlap when the structure is folded. The distal electrode 202 is shown as an uncoated offset region positioned near the middle of the rope wire structure where the loopback occurs.

As further shown in FIG. 2B, the coated portions of the rope wire structure appear with a dotted pattern indicating intermittent coating, while the solid regions represent the uncoated electrode areas. This looped configuration creates a U-shaped parallel rope wire structure that is subsequently wound on a mandrel to produce the helical wire loopback structure electrode 200 with two stimulating proximal electrodes 201 and one distal electrode 202 serving as a collector. The resulting U-shaped parallel rope wire structure is then wound on a core to produce a helical wire structure electrode with a loopback design.

Referring to FIG. 2C, a side view of the helical wire loopback structure electrode 200 in its wound configuration is illustrated. The helical wire loopback structure electrode 200 is shown after the loopback rope wire structure has been wound on a mandrel to create the final helical form. The structure displays a series of alternating dark and light segments along its length, representing the coiled wire rope configuration. Two proximal electrodes 201 are positioned at one end of the helical wire loopback structure electrode 200, serving as stimulating electrodes that are placed on the electrical stimulation target. The distal electrode 202 is located toward the opposite end of the helical wire loopback structure electrode 200, functioning as a collector positioned near or under the skin 3.

With continued reference to FIG. 2C, the helical wire loopback structure electrode 200 exhibits a generally elongated cylindrical profile with a slightly curved overall shape. The wound configuration results from folding a single wire rope structure back upon itself at the distal electrode 202 near the middle of the rope, producing two parallel ropes with offset proximal electrodes 201, which are then wound together to achieve the helical structure shown. This design provides two stimulating proximal electrodes 201 and one distal electrode 202 to serve as the collector within a single integrated helical wire structure.

Two or more helical wire loopback structures are combined by offsetting the loopback rope wire structures at a predetermined distance, placing the loopback rope wire structures in parallel, and then winding the parallel structure around a mandrel. The winding pattern is regular with equally spaced regions between each wire structure in the multi-polar parallel pre-stranding to post winding product, or is intermittently altered where one structure traverses several other structures over the course of single winding. This approach enables the creation of multi-contact helical wire structure electrodes with multiple stimulating electrodes and collectors from combined loopback structures.

Referring to FIG. 3, a side view of the wire structure 400 consisting of the first parallel wire rope structure 402 and the second parallel wire rope structure 403 that are only partially wound together is illustrated. The wire structure 400 includes the first parallel wire rope structure 402 and the second parallel wire rope structure 403 wound into a helical configuration along a portion of the device. At the distal end of the wire structure 400, an electrode 401 is positioned, shown with an alternating pattern indicating exposed uncoated regions for electrical contact with tissue. The wire structure 400 features the unwound section 304 where the first parallel wire rope structure 402 and the second parallel wire rope structure 403 transition from the helical configuration to a non-helical configuration.

With continued reference to FIG. 3, the unwound section 304 extends toward the proximal end of the device. A first non-helical end 404 and a second non-helical end 406 extend from the unwound section 304, with each non-helical end connecting to a single uncoated electrode at the proximal end of the wire structure 400. The first non-helical end 404 and the second non-helical end 406 are shown as parallel elongated structures that taper toward a point at the proximal terminus of the device. This configuration differs from embodiments where all wire rope structures in parallel are wound together from end to end, as the partial winding leaves the unwound section 304 to provide separate electrical connection points for each of the first parallel wire rope structure 402 and the second parallel wire rope structure 403.

As further shown in FIG. 3, the electrode 401 at the distal end provides electrical contact with target tissue, while the first non-helical end 404 and the second non-helical end 406 at the proximal end enable independent electrical connections for each of the parallel wire rope structures. The helical wire structure is formed into a helix with a trailing or front-running tail of rope that is not fully wound into the helix, as demonstrated by the unwound section 304 and the first non-helical end 404 and the second non-helical end 406. Each non-helical end connects to a single uncoated electrode, providing independent electrical pathways for stimulation or sensing through the wire structure 400.

With continued reference to FIG. 3, coating of the wires is achieved by micro coating of individual wires or macro coating of multiple wires. Coating of individual wires with less than 15 micron coatings, and in particular less than 5 micron coatings, maintains mechanical flexibility of the wire structure 400. Coatings are achieved through dip-spin coating of polymers or via vapor deposition methods. The wires are partially coated with inert materials such as Parylene C, fluorinated polymers, or silicones to form lead regions along the first parallel wire rope structure 402 and the second parallel wire rope structure 403, while leaving the electrode 401 and the connection points at the first non-helical end 404 and the second non-helical end 406 uncoated for electrical contact.

Referring to FIG. 4, an image of a manufactured device consistent with the illustration in FIG. 3 is shown. The wire structure 400 comprises two parallel wire rope structures that are only partially wound together to form a helical configuration along a portion of the device. The electrode 401 is positioned at the distal end of the wire structure 400, providing exposed uncoated regions for electrical contact with target tissue during stimulation or sensing operations.

With continued reference to FIG. 4, the first non-helical end 404 and the second non-helical end 406 extend from the proximal end of the wire structure 400. Each of the first non-helical end 404 and the second non-helical end 406 diverges from the main helical body of the wire structure 400, providing separate connection points for electrical interfacing. The first non-helical end 404 connects to a single uncoated electrode at the proximal end of the wire structure 400, and the second non-helical end 406 connects to a single uncoated electrode at the proximal end of the wire structure 400, enabling independent electrical pathways for each of the parallel wire rope structures.

As further shown in FIG. 4, the wire structure 400 demonstrates a configuration where the helical winding does not extend along the entire length of the device. The helical portion of the wire structure 400 provides the stimulating portion of the device with flexibility, bendability, stretchability, compressibility, and pushability characteristics. The helical wire structure is highly flexible and is configured to fold along its longitudinal axis by 10 degrees, 30 degrees, or 180 degrees in any direction as measured from the longitudinal axis. The body's encapsulation response to the helical wire structure is less than 100 microns, compared to encapsulation of prior devices in a range up to 1000 microns.

With continued reference to FIG. 4, the multi-contact helical wire structure has a multitude of contacts, including configurations with 3, 4, 5, 6, 7, 8, 9, 10, 11, or 12 contacts. The first non-helical end 404 and the second non-helical end 406 at the proximal end of the wire structure 400 enable connection to implantable pulse generators or extension cables while maintaining the helical structure for the stimulating portion at the distal end where the electrode 401 interfaces with tissue. This partial winding configuration provides the mechanical and electrical properties of the helical structure at the tissue interface while enabling straightforward electrical connections at the proximal end through the first non-helical end 404 and the second non-helical end 406.

Referring to FIG. 5, a delivery system design configured to deploy the partially helical wire structure 500 is illustrated. The delivery system includes the outer cannula 502 that contains the partially helical wire structure 500. The outer cannula 502 is shown both above and below the partially helical wire structure 500, indicating that the outer cannula 502 surrounds the partially helical wire structure 500 during delivery. The outer cannula 502 is a needle, cannula, catheter, tube, or insert configured to house and guide the partially helical wire structure 500 during insertion into a patient.

With continued reference to FIG. 5, the partially helical wire structure 500 comprises the helical wire structure 504 at one end and the non-helical wire structure 506 at the opposite end. The helical wire structure 504 and the non-helical wire structure 506 are connected at the transition region 503, which marks the boundary between the helical configuration and the non-helical configuration. A contact 501 is positioned at the distal end of the helical wire structure 504, providing an exposed uncoated region for electrical interfacing with target tissue during stimulation or sensing operations.

As further shown in FIG. 5, the multi-contact helical wire structure 505 extends from the helical portion toward the proximal end of the device. An inner sleeve is positioned within the outer cannula 502 and contains the non-helical portion of the device. The inner sleeve extends to the transition region 503 where the helical wire structure 504 transitions to the non-helical wire structure 506. The inner sleeve provides mechanical stabilization for the non-helical wire structure 506 during the insertion procedure.

With continued reference to FIG. 5, upon deployment, the inner sleeve enables pushability for insertion of the entire partially helical wire structure 500 into a patient. Pushability is a mechanical property that is inherent to the helical wire structure 504 due to the coiled configuration, but the non-helical wire structure 506 lacks this inherent pushability without the support of the inner sleeve. The inner sleeve mechanically stabilizes the non-helical wire structure 506 by providing structural support that allows the healthcare professional to advance the entire partially helical wire structure 500 through tissue without buckling or deformation of the non-helical wire structure 506.

As further shown in FIG. 5, delivery of the partially helical wire structure 500 is achieved through curved needles or flexible cannulas. The outer cannula 502 includes configurations such as straight needles with slanted or notched tips, straight needles with side ports, straight needles with side ports and inside flexible tubes intended to exit at a 10 to 90 degree angle from the side port, or curved needles with front or side ports. Delivery is achieved through front-loading of the interfacing bare wire structure regions that do not comprise the lead region, which is placed in parallel to the wire structure delivery cannula. The contact 501 at the distal end of the helical wire structure 504 provides electrical connection capability for interfacing with target tissue once the partially helical wire structure 500 is deployed from the outer cannula 502.

Referring to FIG. 6, the multi-contact helical wire structure 505 with integrated ring connectors is illustrated. The multi-contact helical wire structure 505 comprises a helical portion extending between two ends, where the helical structure is formed from wire rope structures wound together. In this specific example, the multi-contact helical wire structure 505 includes two contacts, though configurations with 3, 4, 5, 6, 7, 8, 9, 10, 11, or 12 contacts are also achievable in this configuration.

With continued reference to FIG. 6, the helical structure extends to the first electrode 510 and the second electrode 520. The first electrode 510 and the second electrode 520 are ring contacts commonly found in spinal and peripheral nerve stimulators. The first electrode 510 and the second electrode 520 allow for connection with implanted pulse generators or extension cables through standard ball seal or equivalent connectors.

As further shown in FIG. 6, Detail A highlights the two electrode ends comprising the first electrode 510 and the second electrode 520, illustrating the alternating pattern of the wire rope structures as the wire rope structures transition from the helical configuration. Detail B displays the first ring connector 511 and the second ring connector 521, which are standard ring connectors to which the wire ropes of the multi-contact helical wire structure 505 are electrically and mechanically connected.

With continued reference to FIG. 6, the first electrode 510 is associated with the first ring connector 511, and the second electrode 520 is associated with the second ring connector 521. This configuration provides independent electrical pathways for each contact. Individual conductors of the multi-contact helical wire structure 505 are connected to each individual cylindrical contact through welding, brazing, or using a conductive adhesive.

As further shown in FIG. 6, the indirect integration of a lead wire to a wire structure is achieved through interweaving and twisting of the lead materials' conductive wire element and the wires of the wire structure, or through the use of welding or brazing approaches. The upper enlarged view shows the complete multi-contact helical wire structure 505 with the helical portion in the center and the electrode regions at each end, demonstrating the transition from the tightly wound helical structure to the ring connector regions.

With continued reference to FIG. 6, the multi-connector embodiment is used for electrical stimulation, or other applications such as radiofrequency ablation. The first ring connector 511 and the second ring connector 521 enable the multi-contact helical wire structure 505 to interface with implantable pulse generators through standard connection mechanisms, providing reliable electrical and mechanical connections for therapeutic applications including neuromodulation and ablation procedures.

Referring to FIG. 7, a magnified view of a distal end of the multi-contact electrode 800 is illustrated. The multi-contact electrode 800 includes three ring contacts arranged along its length, specifically the first ring contact 805, the second ring contact 810, and the third ring contact 815. The first ring contact 805, the second ring contact 810, and the third ring contact 815 are positioned in a linear arrangement along the longitudinal axis of the multi-contact electrode 800, with each ring contact providing an independent electrical interface point for connection to an implantable pulse generator or extension cable.

With continued reference to FIG. 7, the insulative embedding 806 is positioned between the first ring contact 805 and the second ring contact 810, and the insulative molding 811 is positioned between the second ring contact 810 and the third ring contact 815. The insulative embedding 806 and the insulative molding 811 comprise semi-clear plastic material that provides electrical isolation between adjacent ring contacts. The insulative embedding 806 and the insulative molding 811 also provide structural support to maintain the spacing and alignment of the first ring contact 805, the second ring contact 810, and the third ring contact 815 along the multi-contact electrode 800.

As further shown in FIG. 7, the wire rope structure 820 extends from the distal portion of the multi-contact electrode 800 and transitions from a helical configuration to a non-helical configuration as the wire rope structure 820 approaches the ring contacts. The insulative embedding 806 and the insulative molding 811 physically stabilize the wire rope structure 820 during this transition from helical to non-helical form. This stabilization enables proper physical and electrical connections between the wire rope structure 820 and the individual ring contacts, specifically the first ring contact 805, the second ring contact 810, and the third ring contact 815.

With continued reference to FIG. 7, each ring contact is independently associated with and electrically connected to corresponding conductors within the wire rope structure 820. The insulative material separating the cylindrical conductive contacts is achieved through semi-clear plastic embedding or injection molding that separates the ring contacts and stabilizes physically the wire rope structures as the wire rope structures transition from helical to non-helical configurations. This configuration allows each of the first ring contact 805, the second ring contact 810, and the third ring contact 815 to function as an independent electrical pathway for stimulation or sensing operations.

As further shown in FIG. 7, locations of higher stiffness are provided via added glue or heightened tightening via rolling or other means of mechanical stiffening along the longitudinal axis of the wire rope structure, such as the addition of coatings or adhesives 820. Locations of lower stiffness are provided by lesser rolling or lesser compression to allow for predetermined locations of folding during deployment. The wire rope structure 820 is stiff for 2 mm in length followed by 1 mm in lesser mechanical stiffness in some configurations. In other configurations, the wire rope structure 820 is stiff for more than 2 mm, such as 5 mm, 10 mm, or 20 mm, followed by 0.5 mm or more or less than 1 mm in lesser mechanical stiffness. These variations in stiffness along the longitudinal axis of the wire rope structure 820 enable controlled bending and folding behavior during the deployment procedure while maintaining structural integrity, such as at the transition region where the wire rope structure 820 connects to the first ring contact 805, the second ring contact 810, and the third ring contact 815.

Referring to FIG. 8, a perspective view of the electrode assembly 900 comprising the multi-contact electrode 800 connected to the implantable pulse generator 915 is illustrated. The electrode assembly 900 includes the multi-contact electrode 800 and the helical wire structure 910 extending from the multi-contact electrode 800. The multi-contact electrode 800 includes a transparent or semi-transparent housing portion that reveals internal cylindrical components corresponding to ring contacts for electrical interfacing. The helical wire structure 910 extends from the multi-contact electrode 800 as a coiled wire configuration designed for implantation at a target tissue site.

With continued reference to FIG. 8, the implantable pulse generator 915 is shown as an oval-shaped metallic housing with a brushed surface finish. The connector 920 is positioned at the interface between the multi-contact electrode 800 and the implantable pulse generator 915. The connector 920 provides the electrical connection point where the contacts of the multi-contact electrode 800 are electrically attached to the implantable pulse generator 915. This configuration enables the delivery of electrical stimulation from the implantable pulse generator 915 through the helical wire structure 910 to electrodes positioned at the distal end of the electrode assembly 900 for therapeutic applications such as neuromodulation.

As further shown in FIG. 8, the helical wire structure 910 is highly conductive for electric current and also for other forms of energy including radiofrequency current, microwave current, and direct current, as well as ultrasound. The helical wire structure 910 supplies electrical stimulation or blocking energy to neural tissue. The helical wire structure 910 also supplies longer-term electrical blocking energy that induces changes of the pH in the direct vicinity of the electrode to allow for an electrically induced chemical lesion of a tissue target.

With continued reference to FIG. 8, the electrode assembly 900 transfers electrical, thermal, acoustic, mechanical, or electrical energy to initiate a dislodging process from the tissue surrounding the electrode. The time of deployment of the electrode assembly 900 is between 5 and 30 minutes, with a deployment time of 15 minutes being suitable for many applications. The electrode is left percutaneous for a non-chronic duration of less than one year to achieve a direct electrical connection in some configurations.

Referring to FIG. 9, a method for adding a connector to a previously implanted helical wire structure 1001 without complete removal is illustrated. The helical wire structure 1001 is partially removed through the incision 1002 in the skin 3. The incision 1002 provides access to the electrode structure while maintaining the proximal end of the helical wire structure 1001 unmoved within the body. The helical wire structure 1001 extends from beneath the skin 3 through the incision 1002, allowing the distal component under the skin 3 to be partially extracted.

With continued reference to FIG. 9, the elongated contact 1004 is positioned at the collector site. The partial extraction of the helical wire structure 1001 extends the structure back to its non-helical rope wire configuration. The exposed collector 1008 is created after the partially elongated electrode end is cut to length. The exposed collector 1008 provides an uncoated region of the wire rope structure for electrical and mechanical connection to the cylindrical conductive contact 1005.

As further shown in FIG. 9, the cylindrical conductive contact 1005 is applied to the exposed collector 1008 by threading the wire rope structure into the cylinder. The crimp 1006 is applied to the cylindrical conductive contact 1005 to mechanically and electrically secure the cylindrical conductive contact 1005 to the exposed collector 1008. The crimp 1006 applies a crimping force or distance to achieve the mechanical and electrical connection between the cylindrical conductive contact 1005 and the exposed collector 1008.

With continued reference to FIG. 9, an alternative attachment method is depicted where the swage 1007 is applied to the cylindrical conductive contact 1005. The swage 1007 provides a predetermined diameter reduction to achieve mechanical and electrical connection between the cylindrical conductive contact 1005 and the exposed collector 1008. The swage 1007 secures the cylindrical conductive contact 1005 to the exposed collector 1008 through the diameter reduction process.

As further shown in FIG. 9, this process converts the helical wire structure 1001 without complete removal to an electrode that is placed into the socket connector of the implantable pulse generator 915. The subcutaneous pocket for the collector is at a depth of about 1 to 3 mm inside the body. The deployment process partially unwinds some of the strands from the main helical structure and pushes the strands between 1 to 200 microns away, which is mechanically distant enough from the helical structure to be mechanically free to move with the tissue. The wound is closed with sutures, steri strips, or suture glue after the electrode has been placed.

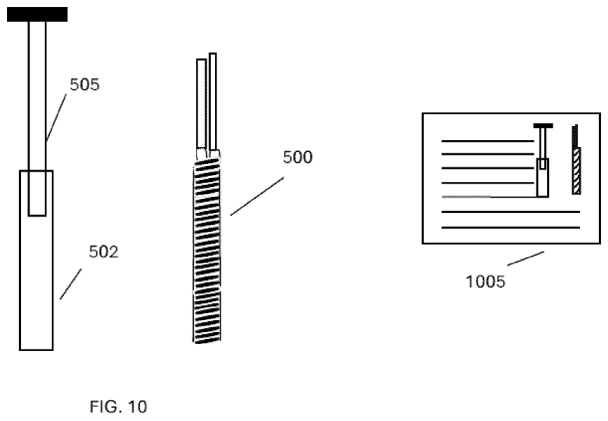

FIG. 10 illustrates a kit for inserting the electrode of the present disclosure. The assembly of all the items into a kit as shown in FIG. 10 makes the insertion and removal of the multi-contact wire structure electrode simple and easy for the user or healthcare professional because the parts are easy to place in the trocar needle and the trocar needle is easily removed leaving the electrode behind in the patient. The kit comprises the user instructions 1005, the trocar 502, the pusher 505 and electrode 500. The distal end of the electrode naturally bunches when the trocar needle is withdrawn forming an anchor for the electrode eliminating the need for the healthcare professional to have to cut or suture the patient. The kit would comprise of an insertion trocar 502 electrode 500, a pusher 505, and electrode 500 has at least one electrode at or near the distal end and at least one contact at or near the proximal end.

The electrode of the current disclosure can be further described as a multi-contact wire structure electrode of the current disclosure, comprising:
a. a plurality of parallel strands of rope wire structures wound into a helical structure;
b. two or more proximal stimulating electrodes formed by uncoated regions of the rope wire structures; and
c. one or more distal connectors for electrical interfacing, wherein the distal connectors are configured for connection to an implantable pulse generator.

The multi-contact wire structure electrode of the current disclosure, wherein the rope wire structures comprise conductive wires selected from the group consisting of gold, silver, platinum, stainless steel, titanium, titanium-nickel, iridium, platinum-iridium, tungsten, platinum-tungsten, and metal alloys.

The multi-contact wire structure electrode of the current disclosure, wherein the conductive wires have a diameter in a range of 2 to 300 microns.

The multi-contact wire structure electrode of the current disclosure, wherein the one or more distal connectors comprise ring connectors configured for connection with a pulse generator, other electronic device or implanted pulse generators through ball seal connectors.

The multi-contact wire structure electrode of the current disclosure, further comprising an insulative material separating adjacent ring connectors from each other.

The multi-contact wire structure electrode of the current disclosure, wherein the insulative material comprises a plastic embedding or injection molding that stabilizes the rope wire structures as the rope wire structures transition from a helical configuration to a non-helical configuration.

The multi-contact wire structure electrode of the current disclosure, wherein the helical structure has an outer diameter within a range of 0.2 to 3.0 mm.

The multi-contact wire structure electrode of the current disclosure, wherein the rope wire structures are formed by 3 to 100 parallel strands of single wires.

The multi-contact wire structure electrode of the current disclosure, wherein the helical structure is configured to fold along a longitudinal axis by up to 180 degrees in any direction.

The multi-contact wire structure electrode of the current disclosure, wherein the rope wire structures comprise an intermittent coating applied along portions of the rope wire structures, the intermittent coating leaving the two or more proximal stimulating electrodes exposed as uncoated regions.

The multi-contact wire structure electrode of the current disclosure, wherein the intermittent coating comprises an inert material selected from the group consisting of Parylene C, fluorinated polymers, and silicones.

The delivery system of the current disclosure can be further described as a delivery system for inserting a partially helical wire structure electrode, comprising:
a. an outer cannula configured to contain a partially helical wire structure electrode having a helical region and a non-helical region; and
b. an inner sleeve positioned within the outer cannula, the inner sleeve configured to contain the non-helical region of the partially helical wire structure electrode and extending to a transition region between the helical region and the non-helical region, wherein the inner sleeve mechanically stabilizes the non-helical region during insertion.

The delivery system of the current disclosure, wherein the outer cannula comprises a needle having a configuration selected from the group consisting of a straight needle with a slanted tip, a straight needle with a notched tip, a straight needle with a side port, and a curved needle with a front port.

The delivery system of the current disclosure, wherein the outer cannula comprises a straight needle with a side port and an inside flexible tube configured to exit at an angle of 10 to 90 degrees from the side port.

The delivery system of the current disclosure, wherein the inner sleeve provides pushability for insertion of the partially helical wire structure electrode into a patient by providing structural support to the non-helical region that prevents buckling during advancement through tissue.

The delivery system of the current disclosure, wherein the partially helical wire structure electrode comprises one or more contacts positioned at a distal end of the helical region for electrical interfacing with target tissue.

The method of the current disclosure can further be described as a method for adding a connector to a previously implanted helical wire structure electrode of the current disclosure, comprising:
a. creating an incision in skin to access a distal portion of the previously implanted helical wire structure electrode;
b. partially extracting the distal portion of the helical wire structure electrode through the incision to extend the helical wire structure electrode to a non-helical rope wire configuration while maintaining a proximal end of the helical wire structure electrode unmoved within a body;
c. cutting the partially extracted non-helical rope wire configuration to a desired length to create an exposed collector;
d. threading the exposed collector into a cylindrical conductive contact; and
e. securing the cylindrical conductive contact to the exposed collector through at least one of crimping or swaging.

The method of the current disclosure, wherein securing the cylindrical conductive contact to the exposed collector through crimping comprises applying a crimping force to the cylindrical conductive contact to mechanically and electrically secure the cylindrical conductive contact to the exposed collector.

The method of the current disclosure, wherein securing the cylindrical conductive contact to the exposed collector through swaging comprises applying a predetermined diameter reduction to the cylindrical conductive contact to mechanically and electrically secure the cylindrical conductive contact to the exposed collector.

The method of the current disclosure, further comprising closing the incision with at least one of sutures, steri-strips, or suture glue after securing the cylindrical conductive contact to the exposed collector.

Referring now to the drawings FIGS. 1-9, and more particularly to FIG. 1A FIG. 1B and FIG. 1C, there is shown an embodiment where three wire rope structures 100 with intermittent coating 119 FIG. 1A having proximal electrodes 101 uncoated on each end and an uncoated offset region 102 near the middle such that when placed in parallel with an offset as shown in FIG. 1B may be wound together as shown in FIG. 1C to achieve a helical wire structure 100 with three independent contacts or electrodes 101 at a spacing dependent on the number of parallel wire rope structures with intermittent coating 119, the offset between the three parallel wire rope structures, and finally the pitch and core diameters of the helical structure achieved through winding together of the wire rope structures.

FIG. 2A, FIG. 2B and FIG. 2. C show a rope wire structure 200 with proximal electrodes 201 uncoated on each end and an uncoated offset region forming distal electrode 202 near the middle. As shown in FIG. 2B the rope wire structure is looped back upon itself with offset proximal electrodes 201 and uncoated offset region forming distal electrode 202. As shown in FIG. 2C the loopback rope wire structure is wound on a mandrel to create the helical wire loopback structure electrode 200 with two stimulating proximal electrodes 201 and one distal electrode 202 to serve as the collector.

FIG. 3 shows a helical wire rope structure 400 consisting of two parallel wire rope structures 402 and 403 that are only partially wound together as to leave an unwound section differing from the embodiment in FIG. 1 where all wire rope structures in parallel are wound together from end to end. Each non-helical ends 404 and 406 of the wire structure 400 only connects to a single uncoated electrode at the proximal end of the wire structure 400 and have electrodes 401 at the distal end and the non-helical ends 404 and 406.

FIG. 4 shows an image of a device manufactures consistent with the illustration in FIG. 3. Where in electrodes 401 are at the distal end and non-helical ends 404 and 406 are at the proximal end of the wire structure 400.

FIG. 5 shows an embodiment of the delivery system design that would be used to deploy the partially helical wire structure 500 in FIGS. 3 and 4, where a trocar needle or outer cannula 502 contains the partially helical wire structures 504 and 506 with one or more contacts 501 with an additional inner sleeve 505 containing the non-helical portion of the device that only extends to the transition region 503 of helical to non-helical structure. Upon deployment, the inner sleeve 505 enables pushability for insertion into a patient of the entire structure that is normally only inherent to the helical wire structure. Once the helical wire structure 500 is deployed in the patient the distal end bunches forming an anchor that prevents the helical wire structure 500 from be pulled out on its own or by a force applied to the proximal end of helical wire structure 500 To remove the helical wire structure 500 the healthcare professional applies a local anesthetic and makes a small incision on the skin above the uppermost portion of the electrode and pulls out the helical wire structure which unzips individual coils of the structure as they release from the tissue first pulling inward towards the center line of the helical wire structure electrode before being pulled outward to exit the body. When a coil 506 is pulled, the outer diameter contracts and pulls the outer surface away from surrounding tissue. This releases the inner encapsulation layers from the outer encapsulation layers one coil at a time, allowing the removal at low overall forces as one loop separates tissues mechanically prior to moving outward instead of prior electrodes and leads that require the attached encapsulation layers to remove along the entire implant at once. The separation from encapsulation lays one coil after the other ensures low removal forces and greatly adds to the reliability of the removal process that is easily performed in a clinic or outpatient procedure without major risk of the helical wire structure electrode rupturing, breaking (or “cutting” as healthcare professionals refer to it) of a lead or electrode structure. The in-built quality to deform one coil at a time and thereby reduce the outer diameter of the helical wire structure electrode, while stretching the total length of the helical wire structure, swaps larger outer diameter for more length during the removal process, thereby reducing the removal forces, similar to how an inclined plane reduces the forces needed to lift an object one small incremental step at a time instead of lifting it all at once. This process of incremental removal of one coil at a time, herein described as “unzipping,” is of great benefit to the healthcare professional conducting the removal process (and the patient) as unzipping reduces the possibility of loose pieces, aids with the release from the encapsulation tissue as well as delicate native tissues and enables exiting the body via its own path or footprint occupied chronically inside the body prior to removal.

FIG. 6 shows a multi-contact helical wire structure 505 (two contacts in this specific example, while a multitude of contacts of 3, 4, 5, 6, 7, 8, 9, 10, 11, or 12 are also possible in this configuration) where the helical structure extends to ring 510 and 520 commonly found in spinal and peripheral nerve stimulators to allow for connection with a pulse generator, other electronic device or implanted pulse generators or their extension cables through standard ball seal or equivalent connectors. Detail A highlights the two electrode 510 and 520 ends and Detail B shows the standard ring connectors 511 and 521 to which the wire ropes of the helical wire rope structure 505 are electrically and mechanically connected to achieve Contact 510 and 520 associated only with Electrode 511 and 521.

FIG. 7 shows a magnified view of a 3 ring contact distal end of the wire structure 800, with a semi-clear plastic embedding or injection molding 806 and 811 that separates the ring contacts 805, 810 and 815, stabilize physically the wire rope structures 820 as they transition from helical to non-helical in order to allow for physical and electrical connections with the individual ring contacts.

FIG. 8 shows the multi-contact electrode 800 from FIG. 7 connected to an implantable pulse generator 915. multi-contact electrode 800 has electrodes 901 and the contacts electrically attached to a pulse generator, other electronic device or implantable pulse generator 915 at connector 920.

FIG. 9 shows a previously implanted helical wire structure 1001 that is partially removed through an incision 1002 in the skin 1003 and elongated contact 104 at the collector site. The cylindrical conductive 1005 contact is crimped 1006 onto the exposed and cut collector 1008. A cylindrical conductive contact is swaged 1007 onto the exposed and cut collector of the helical wire structure electrode 1001.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim.

1. A multi-contact wire structure electrode, comprising:
a plurality of parallel strands of rope wire structures wound into a helical structure;
two or more proximal stimulating electrodes formed by uncoated regions of the rope wire structures; and
one or more distal connectors for electrical interfacing, wherein the distal connectors are configured for connection to a pulse generator.

2. The multi-contact wire structure electrode of claim 1, wherein the rope wire structures comprise conductive wires selected from the group consisting of gold, silver, platinum, stainless steel, titanium, titanium-nickel, iridium, platinum-iridium, tungsten, platinum-tungsten, and metal alloys.

3. The multi-contact wire structure electrode of claim 2, wherein the conductive wires have a diameter in a range of 2 to 300 microns.

4. The multi-contact wire structure electrode of claim 1, wherein the one or more distal connectors comprise ring connectors configured for connection with implanted pulse generators through ball seal connectors.

5. The multi-contact wire structure electrode of claim 4, further comprising an insulative material separating adjacent ring connectors from each other.

6. The multi-contact wire structure electrode of claim 5, wherein the insulative material comprises a plastic embedding or injection molding that stabilizes the rope wire structures as the rope wire structures transition from a helical configuration to a non-helical configuration.

7. The multi-contact wire structure electrode of claim 1, wherein the helical structure has an outer diameter within a range of 0.2 to 3.0 mm.

8. The multi-contact wire structure electrode of claim 1, wherein the rope wire structures are formed by 3 to 100 parallel strands of single wires.

9. The multi-contact wire structure electrode of claim 1, wherein the helical structure is configured to fold along a longitudinal axis by up to 180 degrees in any direction.

10. The multi-contact wire structure electrode of claim 1, wherein the rope wire structures comprise an intermittent coating applied along portions of the rope wire structures, the intermittent coating leaving the two or more proximal stimulating electrodes exposed as uncoated regions.

11. The multi-contact wire structure electrode of claim 10, wherein the intermittent coating comprises an inert material selected from the group consisting of Parylene C, fluorinated polymers, and silicones.

12. A delivery system for inserting a partially helical wire structure electrode, comprising:
an outer cannula configured to contain a partially helical wire structure electrode having a helical region and a non-helical region; and
an inner sleeve positioned within the outer cannula, the inner sleeve configured to contain the non-helical region of the partially helical wire structure electrode and extending to a transition region between the helical region and the non-helical region, wherein the inner sleeve mechanically stabilizes the non-helical region during insertion.

13. The delivery system of claim 12, wherein the outer cannula comprises a needle having a configuration selected from the group consisting of a straight needle with a slanted tip, a straight needle with a notched tip, a straight needle with a side port, and a curved needle with a front port.

14. The delivery system of claim 13, wherein the outer cannula comprises a straight needle with a side port and an inside flexible tube configured to exit at an angle of 10 to 90 degrees from the side port.

15. The delivery system of claim 12, wherein the inner sleeve provides pushability for insertion of the partially helical wire structure electrode into a patient by providing structural support to the non-helical region that prevents buckling during advancement through tissue.

16. The delivery system of claim 12, wherein the partially helical wire structure electrode comprises one or more contacts positioned at a distal end of the helical region for electrical interfacing with target tissue.

17. A method for adding a connector to a previously implanted helical wire structure electrode, comprising:
creating an incision in skin to access a distal portion of the previously implanted helical wire structure electrode;
partially extracting the distal portion of the helical wire structure electrode through the incision to extend the helical wire structure electrode to a non-helical rope wire configuration while maintaining a proximal end of the helical wire structure electrode unmoved within a body;
cutting the partially extracted non-helical rope wire configuration to a desired length to create an exposed collector;
threading the exposed collector into a cylindrical conductive contact; and
securing the cylindrical conductive contact to the exposed collector through at least one of crimping or swaging.

18. The method of claim 17, wherein securing the cylindrical conductive contact to the exposed collector through crimping comprises applying a crimping force to the cylindrical conductive contact to mechanically and electrically secure the cylindrical conductive contact to the exposed collector.

19. The method of claim 17, wherein securing the cylindrical conductive contact to the exposed collector through swaging comprises applying a predetermined diameter reduction to the cylindrical conductive contact to mechanically and electrically secure the cylindrical conductive contact to the exposed collector.

20. The method of claim 17, further comprising closing the incision with at least one of sutures, steri-strips, or suture glue after securing the cylindrical conductive contact to the exposed collector.
